(19) United States
(12) Patent Application Publication
(10) Pub. No.: US 2018/0252674 A1
(43) Pub. Date: Sep. 6, 2018

(54) HANDHELD APPARATUS FOR TESTING A SAMPLE OF PREPARED FOOD FOR ALLERGENS AND/OR FOOD INTOLERANCE INGREDIENTS

(71) Applicant: Pro-Ino Development APS, Herning (DK)

(72) Inventor: Jens Hansen, Dronninglund (DK)

(21) Appl. No.: 15/759,483

(22) PCT Filed: Sep. 13, 2016
(86) PCT No.: PCT/EP2016/071544

§ 371 Date: Mar. 12, 2018

(30) Foreign Application Priority Data
Sep. 13, 2015 (DK) PA 2015 00540

(51) Int. Cl. G01N 27/416; G01N 33/02; G01N 27/28

(52) U.S. Cl. CPC G01N 27/416; G01N 27/286; G01N 33/02

(57) ABSTRACT

A handheld apparatus for testing a sample of prepared food for allergens and/or food intolerance ingredients. An electronic identification tag comprising information on the type of disposable electrochemical test unit is embedded in the disposable electrochemical test unit.

### TECHNICAL FIELD OF THE INVENTION

The present invention relates to testing prepared food for allergens and/or food intolerance ingredients. More particularly, the present invention relates to handheld apparatuses for testing a sample of prepared food for allergens and/or food intolerance ingredients.

### BACKGROUND OF THE INVENTION

The medical sequelae of food allergies and food intolerances can range from a mild reaction, such as nasal congestion to anaphylactic shock, and potential death. Non-limiting examples of foods which may elicit an allergic reaction or a food intolerance-associated reaction include nuts such as peanuts and tree nuts (such as almonds, cashews, hazels walnuts and pecans), wheat (gluten) and in particular gluten, dairy (e.g. milk (lactose)), eggs, fish and shellfish.

People with severe food allergies/intolerance typically rely on trust and reputation when it comes to prepared food and possible allergens and/or food intolerance ingredients therein. If available, product labels are carefully scrutinized, and prepared food products that are labelled in a way that meets the consumer's confidence are selected.

In cases where a person with food allergy visits a restaurant, the person must rely on the report of staff about whether the food is prepared in contact with an allergen and/or food intolerance ingredient.

It is therefore desirable to provide an easy-to-use method that can identify the presence of an allergen and/or food intolerance ingredients (e.g., lactose) in prepared food to provide additional confidence to someone suffering from severe food allergies and intolerances, in a context, where the source of food is not trusted.

Self-testing of prepared food in restaurants, at home, or at parties for allergens and/or food intolerance ingredients will aid those suffering from allergies to live more safely. Often, it may be necessary to test for various allergens and/or food intolerance ingredients. Hence, there is a need for a system capable of handling multiple types of tests.

### SUMMARY OF THE INVENTION

A first aspect relates to a handheld apparatus configured for testing a sample of prepared food for multiple types of allergens and/or food intolerance ingredients; the apparatus comprising:
a handheld test meter adapted for receiving a disposable electrochemical test unit; and
a disposable electrochemical test unit configured for electrochemical sensing of an allergen and/or food intolerance ingredient;
   wherein the disposable electrochemical test unit comprises a connector configured for connecting with the handheld test meter;
   wherein an electronic identification tag is embedded in the disposable electrochemical test unit, the electronic identification tag comprising information on the type of disposable electrochemical test unit;
   wherein the handheld test meter is configured to read the electronic identification tag, and in response to its information on the type of disposable electrochemical test unit, configured to detect and process the electrochemical signal generated by the disposable electrochemical test unit during use to provide an indication of presence and/or amount of allergen and/or food intolerance ingredient to the user.

A second aspect relates to a disposable electrochemical test unit adapted for electrochemical sensing of an allergen and/or food intolerance ingredient; the disposable electrochemical test unit being configured for electrochemical sensing of an allergen and/or food intolerance ingredient;

wherein the disposable electrochemical test unit comprises a connector configured for connecting with a handheld test meter;
wherein an electronic identification tag is embedded in the disposable electrochemical test unit, the electronic identification tag comprising information on the type of disposable electrochemical test unit.

A third aspect relates to a handheld test meter adapted for receiving a disposable electrochemical test unit; wherein the handheld test meter is configured to read an electronic identification tag embedded in a disposable electrochemical test unit, and in response to its information on the type of disposable electrochemical test unit, configured to detect and process the electrochemical signal generated by the disposable electrochemical test unit during use to provide an indication of presence and/or amount of allergen and/or food intolerance ingredient to the user.

### DETAILED DESCRIPTION OF THE INVENTION

A first aspect relates to a handheld apparatus configured for testing a sample of prepared food for multiple types of allergens and/or food intolerance ingredients; the apparatus comprising:
a handheld test meter adapted for receiving a disposable electrochemical test unit; and
a disposable electrochemical test unit configured for electrochemical sensing of an allergen and/or food intolerance ingredient;
   wherein the disposable electrochemical test unit comprises a connector configured for connecting with the handheld test meter;
   wherein an electronic identification tag is embedded in the disposable electrochemical test unit, the electronic identification tag comprising information on the type of disposable electrochemical test unit;
   wherein the handheld test meter is configured to read the electronic identification tag, and in response to its information on the type of disposable electrochemical test unit, configured to detect and process the electrochemical signal generated by the disposable electrochemical test unit during use to provide an indication of presence and/or amount of allergen and/or food intolerance ingredient to the user.

Disclosed herein is a handheld apparatus for testing a sample of prepared food for allergens and/or food intolerance ingredients. The apparatus comprises a handheld test meter adapted for receiving a disposable electrochemical test unit.

The handheld apparatus also comprise a disposable electrochemical test unit adapted for electrochemical sensing of an allergen and/or food intolerance ingredient. The handheld test meter work in conjunction with the disposable electrochemical test unit that comprises at least two electrodes (working and counter electrodes) disposed within the receptacle.

The handheld test meter contains the electronics for applying the potential between the working and counter electrodes in the disposable electrochemical test unit, observing the current, and generating a determination of allergen and/or food intolerance ingredient and conveying it to the user. An indication of the presence or absence of detectable amounts of allergen and/or food intolerance ingredient in the sample is generated based on the observed current. Detection of allergen and/or food intolerance ingredient in a sample of prepared food refers to the qualitative, semi-quantitative or quantitative determination of whether an allergen and/or food intolerance ingredient is present in a sample at detectable levels. Qualitative analysis provides merely a yes/no answer as to whether there is detection. Semi-quantitative provides an indication of amount, but with high granularity, for example as presented through a series of lights where the number of lights illuminated indicates the range into which a value falls. Quantitative analysis provides a numerical value for the amount of allergen and/or food intolerance ingredient in the measured sample.

In order to handle different types of electrochemical tests, the handheld test meter is configured to adjust to such situations by running a specific application for each specific electrochemical test. The information about which specific electrochemical test application there is to be run is provided in the disposable electrochemical test unit by an electronic identification tag embedded therein.

In one or more embodiments, the electronic identification tag is a passive RFID tag (Radio Frequency Identification), preferably a passive NFC (Near Field Communication) tag.

Passive RFID tags, such as a passive NFC tag, do not have their own power source. Instead, they are powered by the electromagnetic energy transmitted from the RFID reader. Because the radio waves must be strong enough to power the tags, passive RFID tags have a read range from near contact and up to 25 meters. For the present application, it is preferred that the RFID tags are configured to have a read range from direct contact (0 mm) up to a few millimeters, such as within the range of 0-2 mm. This is to avoid confusion when several disposable electrochemical test units are in close proximity of the handheld apparatus. The NFC technology is suitable for such use.

In one or more embodiments, the RFID tag is configured to have a read range within the range of 0-20 mm, such as within the range of 1-15 mm, preferably within the range of 0-5 mm.

In order to secure the correct position between the electronic identification tag and its reader, the electronic identification tag is, in one or more embodiments, embedded in the connector.

In one or more embodiments, the handheld test meter comprises an insertion hole comprising a clamping section; and wherein the connector comprises an engagement groove adapted to be engaged with an engagement member in the clamping section. In one or more embodiments, the engagement member is an engagement click member. The sound of a “click” reassures the user that the disposable electrochemical test unit is correctly positioned within the insertion hole of the handheld test meter.

In one or more embodiments, the electronic identification tag is embedded in the engagement groove, and wherein the engagement member comprises an electronic identification tag reader.

In one or more embodiments, the wherein the handheld test meter comprises an insertion hole comprising a clamping section; and wherein the connector comprises an engagement member adapted to be engaged with an engagement groove in the clamping section. In one or more embodiments, the engagement member is an engagement click member. The sound of a “click” reassures the user that the disposable electrochemical test unit is correctly positioned within the insertion hole of the handheld test meter.

In one or more embodiments, the electronic identification tag is embedded in the engagement groove, and wherein the engagement member comprises an electronic identification tag reader.

Because it can be very important to know the presence and/or concentration of allergen and/or food intolerance ingredient in prepared food, particularly for people with food allergy or intolerance, a handheld apparatus has been developed by the inventor to enable the average person to sample and test a prepared food product for determining its allergen and/or food intolerance ingredient concentration at any given time.

In one or more embodiments, the disposable electrochemical test unit comprises a receptacle (sample receiving chamber) for a sample of prepared food.

In one or more embodiments, the disposable electrochemical test unit also comprises a reagent composition disposed in the receptacle. The reagent composition may in one or more embodiments comprise an active redox enzyme effective to oxidize or reduce the allergen and/or food intolerance ingredient. A “redox enzyme” refers to an enzyme that catalyzes oxidation or reduction of the allergen and/or food intolerance ingredient to be determined. Exemplary redox enzymes are oxidases such as glucose oxidase, and dehydrogenases such as glucose dehydrogenase or lactate dehydrogenase. The redox enzyme has an active form prior to interaction with the allergen and/or food intolerance ingredient, and an inactive form. In the case of an oxidase, for example, the active form is oxidized and the inactive form is reduced. As will be apparent, the specific enzyme is selected based on its specificity for the allergen and/or food intolerance ingredient to be analyzed. Non-limiting examples of suitable enzymes include glucose oxidase, fructose dehydrogenase, glucose dehydrogenase, alcohol oxidase, lactate oxidase, cholesterol oxidase, xanthine oxidase, and amino acid oxidases.

In one or more embodiments, the reagent composition disposed in the receptacle comprises an electron transfer reagent. An “electron transfer reagent” refers to a compound other than a redox enzyme that receives and donates electrons to and/or from another chemical species or to and/or from an electrode.

In one or more embodiments, the reagent composition disposed in the receptacle comprises a mediator compound. A “mediator compound” refers to an electron transfer reagent that in use reacts with the inactive enzyme to regenerate active enzyme. The mediator compound may also transfer electrons to or from the electrodes although this is not required.

In one or more embodiments, the reagent composition disposed in the receptacle comprises a shuttle compound. A “shuttle compound” refers to an electron transfer reagent that in use transfers electrons to and from the electrodes. In some embodiments of the invention, the shuttle is reduced by transfer of an electron from one electrode and oxidized by transfer of an electron to the other electrode. In other embodiments, the shuttle can also be reduced (or oxidized) by transfer of an electron from the reduced mediator (or to the oxidized mediator). In this instance, in one preferred embodiment, the mediator itself does not react at the electrodes.

In some embodiments, the mediator compound does not transfer electrons directly to or from the shuttle compound. In other embodiments, electron transfer between mediator and shuttle in solution can happen and in other embodiments electron transfer between mediator and shuttle is the most significant method of transferring electrons from the mediator.

The allergen and/or food intolerance ingredient current generated by the redox processes is detected by the handheld test meter, and converted into an allergen and/or food intolerance ingredient concentration reading using an algorithm that relates the test current to an allergen and/or food intolerance ingredient concentration via a simple mathematical formula known within the art of electrochemical testing.

In one or more embodiments, the disposable electrochemical test unit comprises a diluent and/or solvent reservoir in liquid communication with the receptacle. This is an advantage when the sample of prepared food is too viscous/thick for the reagent composition to mix with the sample of prepared food; or for the situation where the sample of prepared food needs to be solubilized prior to testing.

In one or more embodiments, the diluent and/or solvent reservoir is configured as a foil sealed reservoir, a squeeze to burst packet, a peel apart welded compartment, or a squeeze to burst line sealed compartment.

In one or more embodiments, the disposable electrochemical test unit comprises a receptacle for a sample of prepared food, and a diluent and/or solvent reservoir in liquid communication with the receptacle.

In one or more embodiments, the receptacle is configured as a scoop, corer, spoon or pipette. This is an advantage for better sampling of the prepared food.

A second aspect relates to a disposable electrochemical test unit adapted for electrochemical sensing of an allergen and/or food intolerance ingredient; the disposable electrochemical test unit being configured for electrochemical sensing of an allergen and/or food intolerance ingredient;

wherein the disposable electrochemical test unit comprises a connector configured for connecting with a handheld test meter;
wherein an electronic identification tag is embedded in the disposable electrochemical test unit, the electronic identification tag comprising information on the type of disposable electrochemical test unit.

A third aspect relates to a handheld test meter adapted for receiving a disposable electrochemical test unit; wherein the handheld test meter is configured to read an electronic identification tag embedded in a disposable electrochemical test unit, and in response to its information on the type of disposable electrochemical test unit, configured to detect and process the electrochemical signal generated by the disposable electrochemical test unit during use to provide an indication of presence and/or amount of allergen and/or food intolerance ingredient to the user.

It should be noted, that embodiments and features described in the context of one of the aspects of the present invention also apply to the other aspects of the invention.

1. A handheld apparatus configured for testing a sample of prepared food for multiple types of allergens and/or food intolerance ingredients, the apparatus comprising:
a handheld test meter adapted for receiving a disposable electrochemical test unit; and
a disposable electrochemical test unit configured for electrochemical sensing of an allergen and/or food intolerance ingredient;
wherein the disposable electrochemical test unit comprises a connector configured for connecting with the handheld test meter;
wherein an electronic identification tag is embedded in the disposable electrochemical test unit, the electronic identification tag comprising information on the type of disposable electrochemical test unit;
wherein the handheld test meter is configured to read the electronic identification tag, and in response to its information on the type of disposable electrochemical test unit, configured to detect and process the electrochemical signal generated by the disposable electrochemical test unit during use to provide an indication of presence and/or amount of allergen and/or food intolerance ingredient to the user.

2. A handheld apparatus according to claim 1, wherein an electronic identification tag is embedded in the connector.

3. A handheld apparatus according to claim 2, wherein the handheld test meter comprises an insertion hole comprising a clamping section; and
wherein the connector comprises an engagement groove adapted to be engaged with an engagement member in the clamping section.

4. A handheld apparatus according to claim 3, wherein the electronic identification tag is embedded in the engagement groove, and wherein the engagement member comprises an electronic identification tag reader.

5. A handheld apparatus according to claim 2, wherein the wherein the handheld test meter comprises an insertion hole comprising a clamping section; and
wherein the connector comprises an engagement member adapted to be engaged with an engagement groove in the clamping section.

6. A handheld apparatus according to claim 5, wherein the electronic identification tag is embedded in the engagement groove, and wherein the engagement member comprises an electronic identification tag reader.

7. A handheld apparatus according to claim 1, wherein the disposable electrochemical test unit comprises a receptacle for a sample of prepared food configured to control the total volume of the sample of prepared food to a predetermined volume.

8. A handheld apparatus according to claim 1, wherein the disposable electrochemical test unit comprises a receptacle for a sample of prepared food, and a diluent and/or solvent reservoir in liquid communication with the receptacle.

9. A disposable electrochemical test unit adapted for electrochemical sensing of an allergen and/or food intolerance ingredient, the disposable electrochemical test unit being configured for electrochemical sensing of an allergen and/or food intolerance ingredient;
wherein the disposable electrochemical test unit comprises a connector configured for connecting with a handheld test meter; and
wherein an electronic identification tag is embedded in the disposable electrochemical test unit, the electronic identification tag comprising information on the type of disposable electrochemical test unit.

10. A disposable electrochemical test unit according to claim 9, wherein an electronic identification tag is embedded in the connector.

11. A disposable electrochemical test unit according to claim 10, wherein the connector comprises an engagement groove adapted to be engaged with an engagement member in an insertion hole in a clamping section of a handheld test meter.

12. A disposable electrochemical test unit according to claim 11, wherein the electronic identification tag is embedded in the engagement groove.

13. A handheld test meter adapted for receiving a disposable electrochemical test unit, wherein the handheld test meter is configured to read an electronic identification tag embedded in a disposable electrochemical test unit, and in response to its information on the type of disposable electrochemical test unit, configured to detect and process the electrochemical signal generated by the disposable electrochemical test unit during use to provide an indication of presence and/or amount of allergen and/or food intolerance ingredient to the user.

14. A handheld test meter according to claim 13, further comprising an insertion hole comprising a clamping section with an engagement member adapted to be engaged with an engagement groove of a disposable electrochemical test unit.

15. A handheld test meter according to claim 14, wherein the engagement member comprises an electronic identification tag reader.
